(19) United States
(12) Patent Application Publication
(10) Pub. No.: US 2010/0069824 A1
(43) Pub. Date: Mar. 18, 2010

(54) Photodynamic hyperthermic chemotherapy of cancer and therapeutic system therefor

(76) Inventors: Yoshiharu Okamoto, Tottori-shi (JP); Saburo Minami, Tottori-shi (JP); Yoshimitsu Abiko, Chiyoda-ku (JP); Satoshi Otsuka, Southborough, MA (US); Nobuhiko Ogawa, Inagishi (JP); Masaru Matsui, Inagishi (JP)

Correspondence Address: WENDEROTH, LIND & PONACK, L.L.P., 1030 15th Street, N.W.,, Suite 400 East, Washington, DC, 20005-1503, US

(21) Appl. No.: 12/461,758

(22) Filed: Aug. 24, 2009

(30) Foreign Application Priority Data
Sep. 18, 2008 (JP) 2008-239476

(51) Int. Cl. A61M 37/00

(52) U.S. Cl. 604/21

(57) ABSTRACT

A photodynamic hyperthermic chemotherapy of cancer comprising topically injecting a photosensitive dye agent, specifically indocyanine green, in the form of an acidic liquid formulation to a tumor or cancer tissue, or a surgically removed site thereof of a patient, and irradiating the tissue or site with light having such an output wavelength that the photosensitive dye agent absorbs the light and generates heat; and an apparatus and a system for photodynamic hyperthermic chemotherapy of cancer comprising a light source and a probe having a light introducing portion capable of topically irradiating a tumor or cancer tissue or a surgically removed site thereof of a patient with light derived from the light source by a continuous wave or a pulse wave at an output wavelength of 600 to 1600 nm and an output of 5000 mW or more.

### BACKGROUND OF THE INVENTION

1. Field of the Invention

The present invention relates to a photodynamic hyperthermic chemotherapy of cancer and a therapeutic system therefor. More specifically, it relates to a photodynamic hyperthermic chemotherapy of a tumor or cancer of animals including human beings using a photosensitive dye agent, an apparatus used therefor, and the like.

2. Description of the Related Art

Recently, as the span of life of animals such as pet animals has become longer, animals suffering from various diseases have been increased. Cancer is one of such diseases. Recently, keepers who consider their pet animals as a member of their family have been rapidly increased. Therefore, treatment of cancer, specifically advanced treatment of cancer is demanded. Namely, the main three treatment methods against cancers of animals except human beings include, as with in human beings, surgical operation, chemotherapy and radiation therapy. However, in the field of veterinary, radiation therapy is performed only in limited facilities in Japan. Under these circumstances, cancer that cannot be treated by these main three treatment methods has been rapidly increased. Therefore, it is desired to rapidly investigate the “fourth treatment method of cancer” in the field of medicine as well as in the field of veterinary.

Chem. Mater, Vol. 19, No. 6 pp. 1277-1284 (Mar. 30, 2007) discloses synthesis of capsules of aggregated nanoparticles containing indocyanine green (hereinafter sometimes abbreviated as ICG), which generates heat upon reacting to light, and suggests using the capsules in photothermal therapy. Further, Journal of Clinical and Experimental Medicine (Igaku no Ayumi), Additional Volume, December, pp. 89-92 (Dec. 15, 1999) discloses hyperthermic chemotherapy using a magnetic induction tissue heating system (IHS) against VX-7-transplanted tongue tumor in rabbits, performs a combination therapy of hyperthermia using IHS and chemotherapy using cisplatin and peplomycin, and suggests that the hyperthermic chemotherapy using IHS can be expected to be a treatment method aiming at improvement of QOL.

Furthermore, photodynamic therapy (hereinafter sometimes abbreviated as PDT), hyperthermia (hereinafter sometimes abbreviated as HT) and the like are also known.

PDT is a therapy using a photosensitive material having affinity for tumors, in which a chemical reaction is caused at a specific light wavelength to generate active oxygen. The photosensitive material having affinity for tumors has a characteristic that it naturally accumulates more on abnormal cells such as cancer than on normal tissues, and the normal tissues excrete the photosensitive material having affinity for tumors in a shorter time than the abnormal tissues do. The principle of this therapy is such that the photosensitive material having affinity for a tumor is administered to a subject to be treated (patient), the patient waits for several hours to several days, and light having a specific wavelength is irradiated to selectively generate activate oxygen in abnormal cells and the abnormal cells can be killed by the activated oxygen. As the photosensitive material having affinity for tumors, porphyrin, Photofrin, BPD-MA, NPe6, ATX-S10, SnET2, ALA, LuTex and the like are known.

JP 2006-56807 A discloses a formulation containing a photosensitive material (a photosensitizing compound) that is used for PDT. The document also discloses a method including delivering a photosensitive material that does not show affinity for tumors to the vicinity of abnormal cells such as cancer with an angiocatheter or the like, and also suggests combination use with surgery, radiation therapy, chemotherapy and HT.

However, in conventional PDT, a patient suffers from solar photosensitivity for several days to several weeks after injection (mainly by systemic administration) of the photosensitive material, and thus the patient is forced to live with avoiding sunlight. Therefore, there are problems that the method is complex since a medical institution requires time for managing a patient and that QOL of the patient is decreased. Furthermore, since the method utilizes “natural” accumulation of the photosensitive material on the abnormal tissue, there is a problem that it is difficult to control the amount of the photosensitive material actually accumulated on the abnormal tissue.

HT is a treatment using a temperature from 40 to 45° C., which aims at killing tumor cells by utilizing the difference in heat resistance between a normal tissue and a tumor tissue. Namely, if abnormal cells such as cancer form a tumor, blood flow becomes insufficient and lactic acid is produced in the tumor site, whereby the tumor site becomes acidic. Cells put in a higher acidic environment have higher temperature sensitivity, and the cells are killed under a high temperature environment. It is known that cells generally have no control in their recovery function in an environment of 42° C. or higher, and if an environment at a temperature of from 42 to 43° C. is given to a tissue containing a tumor, heat denaturation of protein in the cells is caused. The normal tissue repairs heat denaturation by increasing blood flow and become resistant against heat, whereas the tumor tissue cannot proceed repair of heat denaturation and is destroyed since it does not have any systemic blood vessel tissue.

As techniques for controlling the temperature of the tissue for performing HT, techniques for topically heating the vicinity of an affected site by an electromagnetic or dielectric means, techniques for topically dielectric heating by high frequency wave, use of ultrasonic wave, microwave, laser and the like, and techniques of temperature sensors for “controlling the state of the objective site” have been largely suggested.

However, conventional HT has a problem that the system used is relatively large and expensive. Further, HT also have a problem that control of a specific acidic environment is difficult since realization of the acidic environment is depending on natural occurrence due to the difference in blood flows in tissues.

U.S. Pat. No. 6,768,925 describes that HT enhances the effect of chemotherapy. Furthermore, JP 3-41950 A discloses a technique for applying HT using an endoscope. However, the technique has a problem that the observation distance is changed during treatment since it does not have any means for restricting a tip portion of the endoscope, i.e., an objective lens, and an observation site, i.e., a heating site.

### SUMMARY OF THE INVENTION

One object of the present invention is to provide photodynamic hyperthermic chemotherapy (hereinafter sometimes abbreviated as PHCT) as the “fourth treatment method of cancer”.

Another object of the present invention is to provide an apparatus used for PHCT.

Still another object of the present invention is to provide a system for PHCT.

These and other objects as well as advantages of the present invention will be apparent to those skilled in the art from the following description and the attached drawings.

In order to achieve the above-mentioned objects, the present inventors have focused on the characteristic of a photosensitive dye agent, specifically ICG, to generate heat upon reacting to light, and have studied a method for treating cancer that overcomes the above-mentioned problems in PDT and HT by using the photosensitive dye agent, intensively. As a result, the present inventors have found that ICG generates not only active oxygen but also heat upon irradiation with a certain wavelength. There is no conventional PDT that focuses on this heat generation. According to the present invention, both effects of PDT and HT are exhibited by using not only the excitation wavelength of. ICG of from 600 to 805 nm but also a broadband frequency near infrared wavelength of from 600 to 1600 nm, preferably 600 to 1300 nm. That is, PDT and HT can be simultaneously performed by active oxygen, an exothermic reaction and radiation heat.

ICG is a material that is broadly used in the field of medicine as a reagent for examining the hepatobiliary system and its safety has already been established. However, any method for treating cancer using this material has not been reported yet.

Although the above-mentioned JP 2006-56807 A suggests combination use of PDT. and HT, it does not suggest any specific constitution.

Accordingly, the present invention provides:

(1) A photodynamic hyperthermic chemotherapy of cancer, which comprises topically injecting a photosensitive dye agent in the form of an acidic liquid formulation into a tumor or cancer tissue, or a surgically removed site thereof of a patient, and irradiating the tissue or site with light having such an output wavelength that the photosensitive dye agent absorbs the light and generates heat;

(2) The photodynamic hyperthermic chemotherapy according to the above (1), wherein the acidic liquid formulation is a liquid formulation of pH 4.0 to 6.8;

(3) The photodynamic hyperthermic chemotherapy according to the above (1), wherein the acidic liquid formulation is an aqueous physiological saline solution;

(4) The photodynamic hyperthermic chemotherapy according to the above (1), wherein the acidic liquid formulation is topically injected while keeping at 43 to 44° C.;

(5) The photodynamic hyperthermic chemotherapy according to the above (1), wherein the output wavelength is 600 to 1600 nm;

(6) The photodynamic hyperthermic chemotherapy according to the above (1), wherein the output wavelength is 600 to 1300 nm;

(7) The photodynamic hyperthermic chemotherapy according to the above (1), wherein the photosensitive dye agent is indocyanine green;

(8) The photodynamic hyperthermic chemotherapy according to the above (1), wherein an anticancer agent is topically injected together with the photosensitive dye agent;

(9) The photodynamic hyperthermic chemotherapy according to the above (1), wherein ethanol is topically injected together with the photosensitive dye agent;

(10) The photodynamic hyperthermic chemotherapy according to the above (8), wherein the anticancer agent is cisplatin;

(11) The photodynamic hyperthermic chemotherapy according to the above (8), wherein the anticancer agent is bleomycin;

(12) The photodynamic hyperthermic chemotherapy according to the above (8), wherein the anticancer agent is paclitaxel;

(13) The photodynamic hyperthermic chemotherapy according to the above (8), wherein the anticancer agent is carboplatin;

(14) The photodynamic hyperthermic chemotherapy according to the above (1), wherein the tissue or site is irradiated plural times with light at an output of 5000 mW or more;

(15) The photodynamic hyperthermic chemotherapy according to the above (1), wherein the patient is a human being;

(16) The photodynamic hyperthermic chemotherapy according to the above (1), wherein the patient is an animal other than a human being;

(17) An apparatus for photodynamic hyperthermic chemotherapy of cancer, which comprises a light source, and a probe having a light introducing portion capable of topically irradiating a tumor or cancer tissue, or a surgically removed site thereof of a patient with light derived from the light source by a continuous wave or a pulse wave at an output wavelength of 600 to 1600 nm and an output of 5000 mW or more;

(18) The apparatus for photodynamic hyperthermic chemotherapy according to the above (17), wherein the light source is a halogen lamp,

(19) A system for photodynamic hyperthermic chemotherapy of cancer by topically injecting an acidic liquid formulation comprising indocyanine green and an anticancer agent in the vicinity of a tumor or cancer tissue, or a surgically removed site thereof of a patient, and irradiating the tissue or site with light having such an output wavelength that indocyanine green is excited with causing an exothermic reaction, which comprises:
at least one light source of the light,
an irradiation means for irradiating the tissue or site with the light for exciting indocyanine green,
a warming means for warming the tissue or site by the exothermic reaction, and
a temperature measuring means for measuring the temperature of the tissue or site;

(20) The system for photodynamic hyperthermic chemotherapy according to the above (19), wherein the light source is common to the irradiation means and the warming means;

(21) The system for photodynamic hyperthermic chemotherapy according to the above (19), wherein the irradiation means and the warming means are combined;

(22) The system for photodynamic hyperthermic chemotherapy according to the above (21), wherein the irradiation means comprises at least one irradiation port of light having a wavelength component for exciting indocyanine green, and a transmitting means for transmitting the light from the light source to the irradiation port; and the warming means shares the irradiation port and the transmitting means of the irradiation means, but irradiates a wavelength component that differs from the wavelength component for exciting indocyanine green to cause the exothermic reaction;

(23) The system for photodynamic hyperthermic chemotherapy according to the above (22), wherein the irradiation port is a hard optical elongated element having a spherical tip and a rear end to which a portion connecting to the transmitting means which is an optical fiber is provided;

(24) The system for photodynamic hyperthermic chemotherapy according to the above (23), wherein the irradiation port has an inner surface in a hemispherical shape and an irradiation aperture for the light introduced by the transmitting means at the bottom portion of the hemisphere;

(25) The system for photodynamic hyperthermic chemotherapy according to the above (22), wherein the system includes an endoscope having a rigid or flexible tube through which the transmitting means of an optical fiber optically connected to the light source, at least one channel to allow entry of instruments or manipulators and a fiberscope for transmitting images are running; the tip portion of the transmitting means is optically connected to a light guide to constitute the irradiation port and the rear end of the transmitting means is connected to the light source; the tip portion of the endoscope has a cylindrical element, which is constituted by a transparent element having a longitudinal length corresponding to the focus distance of the objective lens of the fiberscope; apertures of the channel and the light guide are provided on the inner portion of the cylindrical element; and at least one thermocouples is provided on the tip portion of the cylindrical element;

(26) The system for photodynamic hyperthermic chemotherapy according to the above (25), wherein the endoscope further has at least one channel for reducing air pressure in the channel and the cylindrical element;

(27) The system for photodynamic hyperthermic chemotherapy according to the above (19), which has a circuit for controlling the output of the warming means based on a signal from the temperature measuring means;

(28) The system for photodynamic hyperthermic chemotherapy according to the above (19), which further comprises a means for topically injecting the liquid formulation; and the like.

The PHCT according to the present invention performs topical warming by utilizing exothermic reaction of a photosensitive dye agent upon reacting to light as well as radiation heat of the light (HT effect). This method uses a more simple apparatus and an easier operation than those used in hyperthermia performed in the field of medicine of human beings. Further, it has been confirmed that photosensitive dye agents such as ICG absorb light in a wide range of wavelength to generate active oxygen (PDT effect). Therefore, PHCT using ICG is a treatment method that exhibits not only HT effect but also PDT effect. Furthermore, tumor cells can be injured more strongly by adding a small amount of anticancer agent. Specifically, the treatment effect is enhanced by making the ICG solution acidic and adding a small amount of anticancer agent. In addition, it has been confirmed that ethanol enhances the PDT effect of ICG. A tumor or cancer can be injured more strongly by administrating ethanol to a tissue of the tumor or cancer in advance or administrating ethanol in the form of a mixture with ICG.

### BRIEF DESCRIPTION OF THE DRAWINGS

FIG. 1 is a plan view illustrating a light source in an embodiment of an apparatus used in the present invention.

FIG. 2 is a front view illustrating a hand piece of the apparatus in FIG. 1.

FIG. 3 is a side view illustrating the hand piece of FIG. 2.

FIG. 4 is a side view illustrating a probe having a light introducing portion to be attached to the tip of the hand piece.

FIG. 5 is a schematic overall view illustrating use of one embodiment of the system of the present invention.

FIG. 6 is a schematic view illustrating the injection of a liquid formulation in the embodiment in FIG. 5.

FIG. 7 is a schematic view illustrating one embodiment of PHCT of the present invention using the system of FIG. 5.

FIG. 8 is a schematic view including a partial cross-section which illustrates an irradiation means of another embodiment of the present invention.

FIG. 9 is a schematic view illustrating use of another embodiment of PHCT of the present invention.

FIG. 10 is a partial schematic view of the endoscope in the embodiment in FIG. 9, and

FIG. 11 is a schematic view including a partial cross-section illustrating the operation of the endoscope in the embodiment in FIG. 9.

### DETAILED DESCRIPTION OF THE PREFERRED EMBODIMENTS

Hereinafter the present invention will be explained in detail.

Examples of the subject animal of PHCT of the present invention include dog, cat, bird, horse, cattle, rabbit, guinea pig, rat, ferret, hamster, lizard, monkey, squirrel, pig and the like. Further, PHCT of the present invention is also applicable to a human being.

Examples of the tumor or cancer to be treated include superficial cancer such as breast cancer, fibrous sarcoma, squamous cancer, angiosarcoma, malignant melanoma, sebaceous cancer, basal cell cancer, liposarcoma, leiomyosarcoma, rhabdomyosarcoma, obesity liposarcoma, thyroid cancer and lymphoma; internal organ cancer such as stomach cancer, transitional cell carcinoma, hepatocellular cancer, renal cell cancer, adrenal tumor, osteosarcoma, synovial sarcoma, pulmonary cancer and colon cancer; and the like. Furthermore, a treatment effect against lymph or a tumor of blood origin can be expected by injecting a liquid formulation of a photosensitive dye agent into the blood vessel, exposing the blood vessel and irradiating light.

As the photosensitive dye agent to be used, ICG is preferable.

ICG is generally used as an aqueous physiological saline solution for topical administration of from 2 to 5 mg/ml having a pH of from 4.0 to 6.8, preferably a pH of about 5. If desired, an anticancer agent such as cisplatin, carboplatin, bleomycin and paclitaxel can be added to the solution by a small amount of, for example, from about 1/20 to 1/10 of a normal systemic dose in case of cisplatin.

In order to perform PHCT of the present invention, for example, at first, an ICG liquid formulation is topically administered to a tumor or cancer tissue site or a site from where the tumor or cancer tissue site has been surgically removed (hereinafter referred to as a surgically removed sited thereof). Although the administration amount can be appropriately selected according to a particular subject animal, tumor or cancer, usually, it is from 1 to 10 ml a 2 to 5 mg/ml ICG liquid formulation.

A temperature sensor is then placed in or on the tissue, and light is irradiated using an apparatus comprising a suitable light source and an irradiation means while controlling the output of the light source so that the temperature of the skin surface is not raised to 45° C. or higher to avoid burning by monitoring the temperature of the tissue (temperature of the irradiated site, temperature of the body surface and/or deep portion). As the light to be irradiated, light of a radio wave or a microwave having a high output of 5000 mW or more, usually, from 5000 to 10000 mW and an output wavelength of from 600 to 1600 nm, preferably from 600 to 1300 nm. Specifically, when ICG is used, topical warming can be performed by utilizing generation of heat upon absorbing light of 805 nm or more. As the light source, for example, a halogen lamp, an LED, a laser, a discharge lamp, an organic EL or the like can be used. The light can be a continuous wave or a pulse wave and can be selected according to a particular disease case. In order to reach a deeper portion, a pulse wave is desirable.

Since the surface of the site to be irradiated is not a plane surface, light is irradiated with suitably moving the irradiation means or the object to be irradiated so that irradiation of the light can be performed as uniformly as possible.

The irradiation is performed once for from 15 to 40 minutes and is repeated plural times usually from 3 to 5 times at intervals of from 5 to 7 days. Then, it is desirable that the irradiation is further performed 3 to 5 times at intervals of from 10 to 14 days. Thereafter, the irradiation is repeated at intervals of from 1 to 3 months as maintenance therapy.

The apparatus used for performing PHCT of the present invention is not specifically limited, and so-called an infrared treatment apparatus can be used.

The present invention also provides an infrared treatment apparatus that is specifically modified for PHCT. The infrared treatment apparatus of the present invention is an apparatus for photodynamic hyperthermic chemotherapy comprising a light source and a probe having a light introducing portion capable of topically irradiating a naturally occurring tumor or cancer tissue or a surgically removed site thereof in an animal with light derived from the light source by a continuous wave or a pulse wave having an output wavelength of from 600 to 1600 nm at an output of 5000 mW or more.

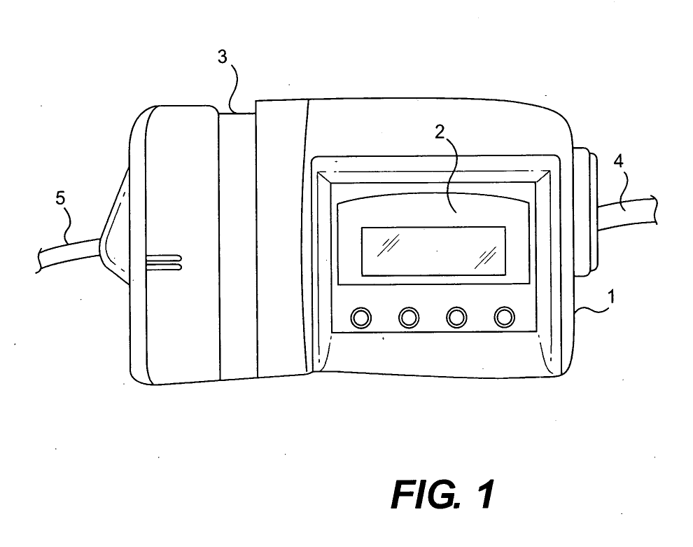

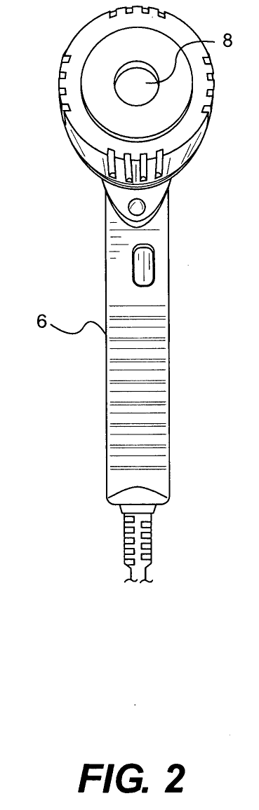

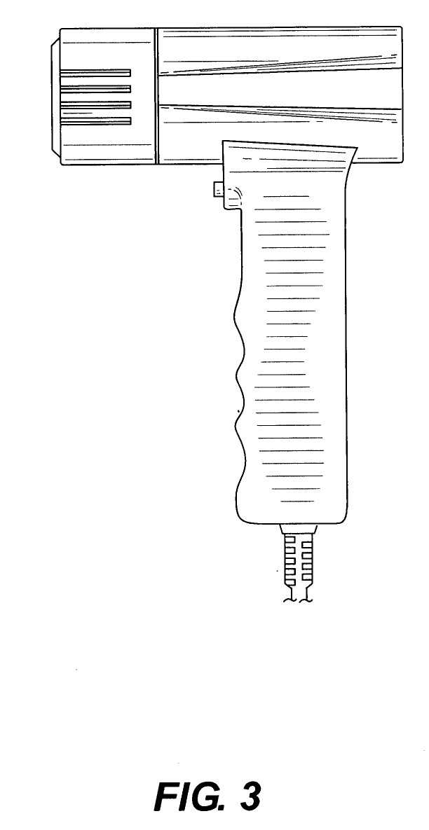

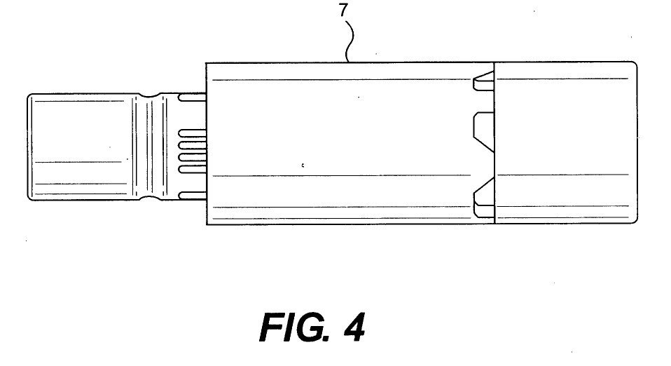

In the attached drawings, FIG. 1 is a plan view illustrating the light source in one embodiment of an apparatus used in the present invention; FIG. 2 is a front view illustrating a hand piece of the apparatus in FIG. 1; FIG. 3 is a side view illustrating the hand piece of FIG. 2; and FIG. 4 is a side view illustrating a probe having a light introducing portion to be attached to the tip of the hand piece.

The apparatus of the present invention generates electrical power to be supplied to a halogen lamp according to irradiation conditions set by a controlling portion 2 in a main body 1, transmits the power through a cable 4 to a halogen lamp contained in a hand piece 6, and irradiates the subject's site through a light introducing portion 7. Further, the main body 1 has a hand piece holder 3 and a power source cable 5 to be connected to a wall socket. An insertion port 8 to which a probe 7 shown in FIG. 4 is to be inserted is provided on the tip portion of the hand piece 6.

When using this apparatus, a power source (not shown) is turn on and adjusted to the irradiation conditions such as specific output, output wavelength, and wave form (continuous wave or pulse wave) by the controlling portion 2.

The light source is not limited to a halogen lamp, and, for example, LED, laser, discharging lamp, or organic EL can also be used.

A timer can be attached to the controlling portion 2 to adjust the irradiation time, and the threshold value of output wavelength can be varied using a variable apparatus, or a single output wavelength can be set. Further, the amount of singlet oxygen generated by PDT effect can be suitably measured by a known method so that the output of the light source can be adjusted. Alternatively, the output of the light source can be adjusted by sensing the temperature of the irradiated site.

The probe can have a movable structure so as to irradiate to the surface to be irradiated uniformly and, for example, a stand can be provided instead of the handpiece to allow irradiation for a long period of time.

Further, in order to allow irradiation to a broader site, a plurality of probes can be mounted, and the probe can have such structure and form that are suitable for the site to be applied such as the nasal cavities, oral cavity, peritoneal cavity and organs.

Next, the system of the present invention for effectively carrying out the PHCT of the present invention will be explained.

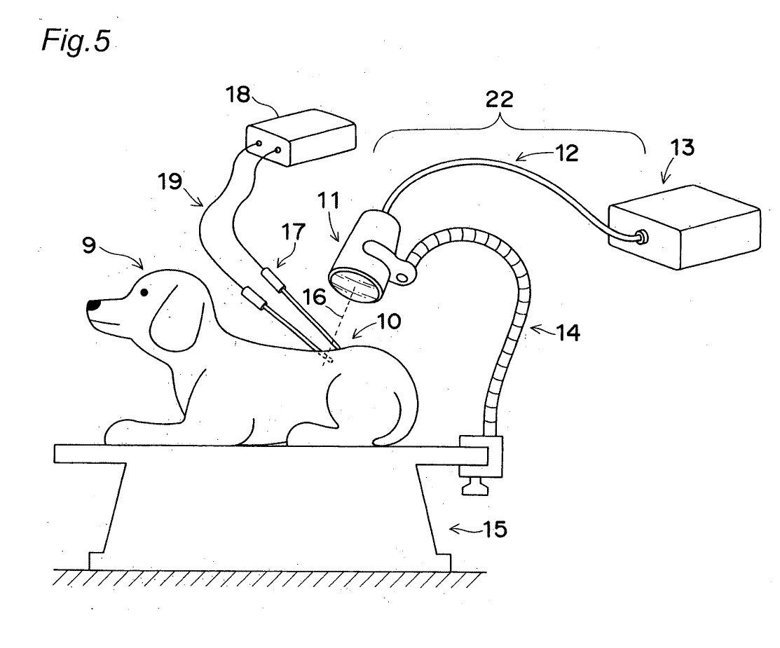

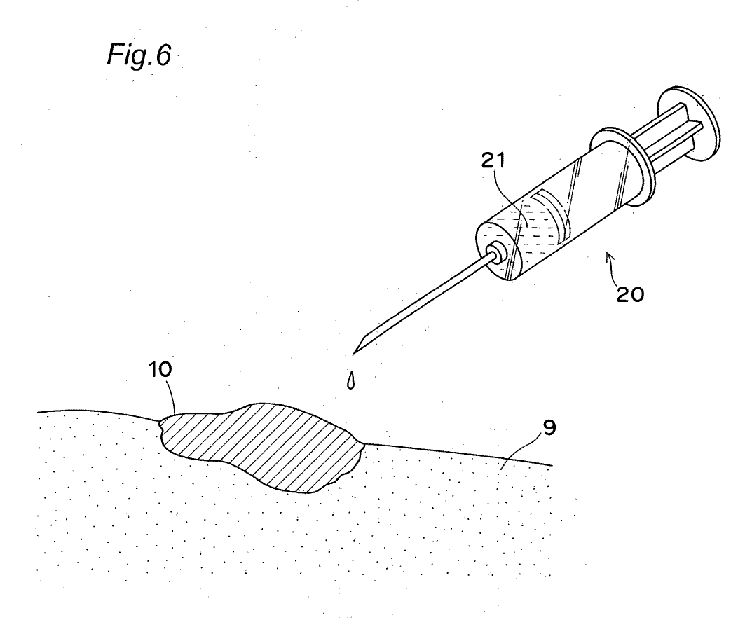

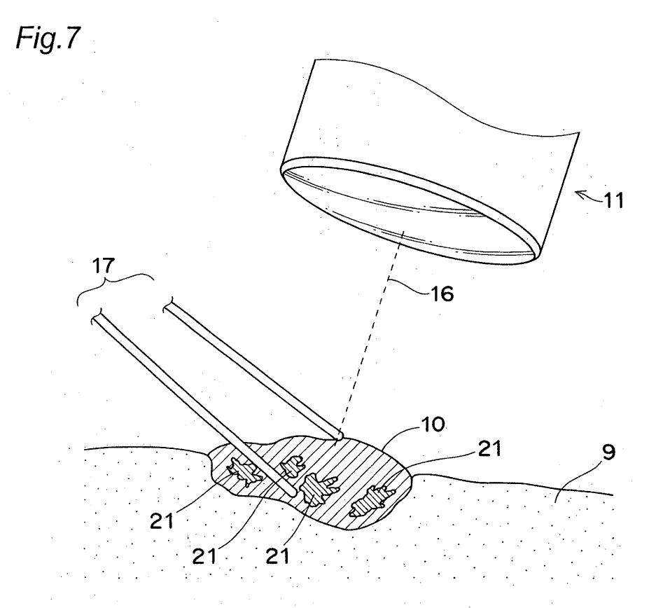

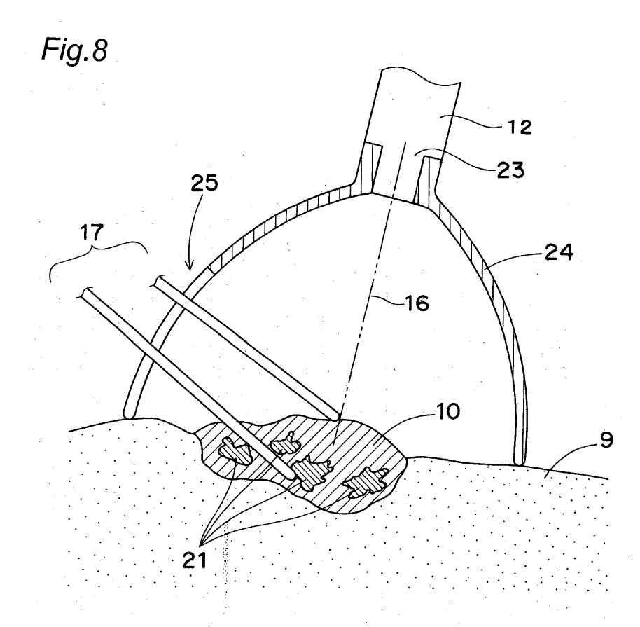

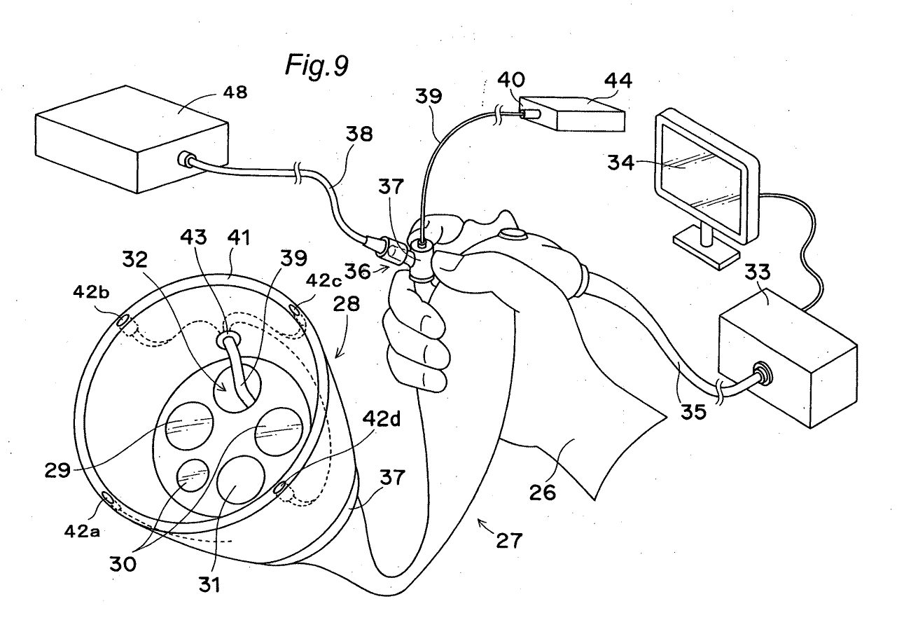

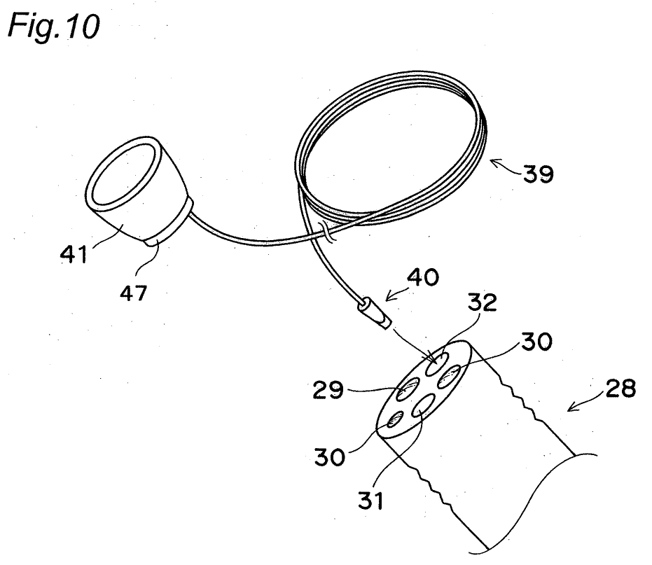

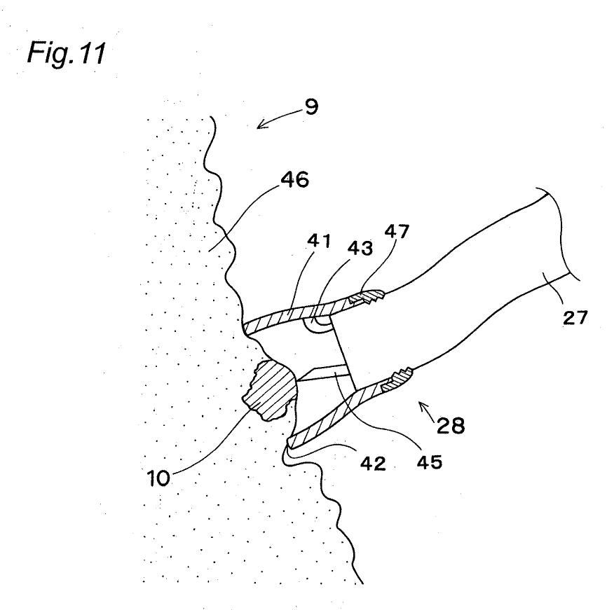

In the attached drawings, FIG. 5 is a schematic overall view illustrating use of one embodiment of the system of the present invention; FIG. 6 is a schematic view illustrating the injection of a liquid formulation in the embodiment in FIG. 5; FIG. 7 is a schematic view illustrating-one embodiment of PHCT of the present invention using the system of FIG. 5; FIG. 8 is a schematic view including a partial cross-section which illustrates an irradiation means of another embodiment of the present invention; FIG. 9 is a schematic view illustrating use of another embodiment of PHCT of the present invention; FIG. 10 is a partial schematic view of the endoscope in the embodiment in FIG. 9; and FIG. 11 is a schematic view including a partial cross-section illustrating the operation of the endoscope in the embodiment in FIG. 9.

The first embodiment of the system of the present invention will be explained with reference to FIGS. 5 to 7.

In this embodiment, a patient (human or animal) 9 having a disease site including an abnormal tissue 10 such as superficial cancer is treated. An irradiation port 11 is optically connected to a light source apparatus 13 via a light guide cable (LG) 12. The light source apparatus 13 is connected to a power source on a wall surface via a power source cable (not shown). The irradiation port 11 is secured at a treatment stage 15 with a holding apparatus 14 whose holding position can be freely changed, and the irradiation light axis 16 is arranged so that irradiation light is fixed on the abnormal tissue 10 in a disease site

The light source apparatus 13 has a luminescence source such as halogen, xenon, metal halide or LED in the inner portion thereof so as to irradiate near infrared ray having a wavelength of from 600 to 1600 nm, preferably 600 to 1300 nm, at least from 600 to 805 nm from the irradiation port 11.

A couple of thermocouple probes 17 are provided. One of the probes is placed on the surface layer of the abnormal tissue 10 and the other is placed in the inner portion of the abnormal tissue 10. Each of the couple of thermocouple probes 17 is electrically connected to a measurement apparatus main body 18 via a cable 19, and the measurement apparatus main body 18 is connected to a power source on a wall via a cable (not shown), which constitute a temperature measuring means of this embodiment.

FIG. 6 shows a means for topically injecting a liquid formulation. In this means, an injection syringe 20 contains an aqueous physiological saline solution 21 consisting of 1 part by volume of ICG, 0.36 part by volume of a weak acidic mixture of acetic acid and saline (preferably at a pH of from 4.0 to 6.8) and 0.04 part by volume of an anticancer agent (cisplatin or bleomycin) (the liquid formulation for topical injection in this embodiment) in an aseptic state.

In FIG. 7, the liquid formulation 21 has been topically injected into the abnormal tissue 10 by the syringe 20.

As shown in FIG. 5, the irradiation port 11, LG 12 and light source apparatus 13 constitute the irradiation means 22 for irradiating a wavelength component having PDT effect (generation of active oxygen), i.e., an wavelength component of from 600 to 805 nm for exciting ICG of the photosensitive material in the topically injected liquid formulation. Further, the irradiation means 22 has a wavelength component of around 800 nm that accelerates the exothermic reaction of ICG in the topically injected liquid. formulation 21 and simultaneously a wavelength component in the near infrared band of from 800 to 1300 nm generates radiation heat, whereby exhibiting HD effect by heat derived from the exothermic reaction together with irradiation heat, which constitutes a warming means.

In order to perform PHCT with the PHCT system of this embodiment, an operator first topically administrates the liquid formulation by syringe 20 to the abnormal tissue 10 of the disease site (i.e., 21 in FIG. 7). When the light source apparatus 13 is turned on, light having a wavelength of from 600 to 1300 nm is irradiated from the irradiation port 11. The irradiation light axis 16 of the light fixed on the abnormal tissue 10 of the disease site, and the light is irradiated to the abnormal tissue 10 including the normal tissue of the disease site. Since a near infrared ray of from 800 to 1300 nm readily penetrates through the body tissue, it penetrates into the inner portion. The light of around 800 nm transmitted into the inner portion causes the exothermic reaction with ICG injected into the inner portion of the abnormal tissue 10 to generate heat. At the same time, the whole irradiated area is heated by the radiation heat possessed by the near infrared ray itself. Further, the light having a wavelength of from 600 to 805 nm reacts with ICG to generate active oxygen, and provides the abnormal tissue 10 with the active oxygen together with the liquid formulation 21 which has been adjusted to weak acidic. Since the impedance of the couple of thermocouple probes 17 provided on the surface layer and inner portion of the abnormal tissue varies according to the temperature of the tissue on which they are provided, the temperature of the tissue about the probes 17 can be measured by the measurement apparatus 18. The operator can perform irradiation for, for example, 20 minutes with adjusting the position of the irradiation port 11 so that the temperature of the surface layer does not exceed 45° C. by using the measurement apparatus 18 and the temperature of the inner portion of the abnormal tissue 10 is kept at 42 to 43° C. At this time, advantageously, the temperature of the surface layer is cooled by spraying sterilized ethanol with a syringe (not shown).

According to this embodiment, the following effects can be obtained.

1) The embodiment is economical since the liquid formulation for topical injection can be produced by using a combination of inexpensive commercially available liquid drugs.

2) Conventional PDT and HT can be performed at the same time using the light source having a wavelength of from 600 to 1300 nm and the topically injectable liquid formulation comprising ICG.

Hereinafter, the second embodiment of the system of the present invention will be explained with reference to FIG. 8.

The second embodiment does not require the holding apparatus 14 used in the first embodiment. Further, the irradiation port 11 of the irradiation means 22 is different from that of the first embodiment. The other constitutions are the same as those of the first embodiment. In FIG. 8, a bowl-shaped element 24 is provided on the tip portion 23 of the LG 12. The bowl-shaped element 24 has a cutout 25 having a width greater than the outer shape of the couple of the thermocouple probes 17, and the couple of thermocouple probes are provided on the inner portion of the bowl-shaped element 24 from outside of the bowl-shaped element 24 through the cutout 25. A light reflecting element (not shown) is deposited on the inner surface of the bowl-shaped element 24.

In order to perform PHCT with the PHCT system of this embodiment, at first, an operator holds the bowl-shaped element 24 as the irradiation port against the disease site including the abnormal tissue 10. Alternatively, the bowl-shaped element 24 and the thermocouple probes 17 can be directly secured on a patient 9 with, for example, an adhesive tape.

According to this embodiment, the following effects can be obtained.

1) Since the holding apparatus 14 is not required, the system can be constituted more easily.

2) Treatment can be performed more easily since the irradiation port can be directly fixed on the disease site of an animal patient moving around.

Hereinafter the third embodiment of the system of the present invention will be explained with reference to FIGS. 9 to 11.

As shown in FIG. 9, the system includes an endoscope having a tube 27 through which the transmitting means of an optical fiber optically connected to a luminescence (light) source (not shown), channels to allow entry of instruments or manipulators and a fiberscope for transmitting images are running. An observation optical objective lens 29, an illumination optical objective lens 30, a channel port for a treatment instrument 31 and a channel port for return 32 are provided on the tip portion 28 of the endoscope. The observation optical objective lens 29 optically connects to an imaging element through the fiberscope (not shown) so that an optical image at the focus distance can be observed. The imaging element is electrically connected to a control box 33, and the control box 33 is connected to a power source on a wall via a cable (not shown). The control box 33 is electrically connected to a monitor 34. The luminescence source is provided in the inner portion of the control box 33, which is optically connected to the illumination objective lens 30 via a light guide cable (not shown) that is running through an endoscope connect cable 35.

A channel port base end aperture for return 36 is connected to the channel port for return 32 in such a manner that air-tight is maintained. A T-shaped port 37 is connected to the channel port base end aperture for return 36, and a vacuum pump 48 is connected to one port of the T-shaped port 37 via a tube 38 and a temperature measuring cable 39 is removably attached to another port of the T-shaped port 37 by an elastic element in such a manner that air-tight can be maintained. The temperature measuring cable 39 is connected to a temperature measuring apparatus 44 via a connector 40 provided on the base end portion of the temperature measuring cable 39. Another tip portion of the temperature measuring cable 39 comes outside from the endoscope tip portion 28 through the channel connected to the channel port for return 32 and is connected to a protruding portion 43 of the cylindrical inner surface of a transparent cylindrical element 41. A plurality of thermocouples 42a to 42d are provided on the end surface of the cylindrical element 41, and the thermocouples 42a to 42d reach the protruding portion 43 through the inner portion of the cylindrical element 41 as shown by the broken line in FIG. 9 and are electrically connected to the temperature measuring cable 39. The tip end position of the cylindrical element 41 (length or height of the element) substantially corresponds to the focus distance of the observation optical objective lens 29.

In FIG. 10, an elastic rubber 47 having an inner diameter smaller than the outer diameter of the endoscope 27 is integrally provided on the base end portion of the cylindrical element 41. Namely, the cylindrical element 41 is removably attached to the tip portion 28 of the endoscope by the elastic rubber 47. The temperature measuring apparatus 44 (see FIG. 9) measures and displays a temperature by the impedance of the thermocouples, which is electrically connected to the temperature measuring cable 39 via the connector 40, and the temperature measuring apparatus 44 is connected to a power source on a wall via a power source cable (not shown).

In FIG. 11, a puncture needle 45 is removably inserted in a channel port for treatment (not shown) of the tip portion 28 of the endoscope 27. The puncture needle 45 comes out from the channel port for treatment at the tip portion 28 of the endoscope. The puncture needle is connected to a syringe (not shown). The liquid formulation 21 that is similar to that shown in the first embodiment is contained in the syringe. The inner wall portion 46 of the stomach or colon of a patient 9 has an abnormal tissue 10 such as superficial cancer.

In order to perform PHCT with the PHCT system shown in this embodiment, the cylindrical element 41 is connected to the endoscope tip portion 28. As shown in FIG. 10, the connector 40 is first inserted into the channel port for return 32 on the endoscope tip portion 28 and pulled from the channel port base end aperture for return 36 provided on the base end portion of the endoscope (see FIG. 9). The elastic rubber 47 is attached to the outer circumference of the endoscope tip portion 28 so as to cover the tip portion 28 while preventing the temperature measuring cable 39 from slacking. At this time, the attachment can be performed more easily by passing physiological saline into the inner portion of the channel for return.

As shown by FIG. 9, an operator 26 inserts the tip portion 28 of the tube of the endoscope 27 into the stomach or colon in the body via the mouth, nasal cavity or anus of the patient 9. The operator 26 identifies the. abnormal tissue 10 according to the image from the observation optical objective lens 29 displayed on the monitor 34, and tightly attaches the end portion of the cylindrical element 41 on the abnormal cell 10 so as to surround the abnormal cell 10. At this time, the inner portion of the cylindrical element 41 is evacuated by actuating the vacuum pump 48, whereby the endoscope tip portion 28 is tightly attached on the inner wall portion 46. The puncture needle 45 put into the channel for treatment is then projected to puncture the abnormal tissue 10, and the liquid formulation is uniformly injected into the inner portion of the abnormal tissue 10 with the syringe. The puncture needle 45 is picked, and a luminescence source (not shown) provided on the inner portion of the control box 33 is then started to irradiate light having a band frequency of from 600 to 1300 nm from the illumination optical objective lens. Since the thermocouple provided on the tip portion of the cylindrical element 41 is tightly attached on the inner wall portion 46, the temperature of the tightly attached site is displayed by the temperature measuring apparatus 44. The operator controls the temperature by turning on and turning off the luminescence source provided on the control box 33 so that the surface temperature does not exceed 45° C. while monitoring the displayed temperature.

According to this embodiment, the following inherent effects can be obtained.

1) Since PDT and HT can be simultaneously performed, the operation efficiency is more improved than the case where the treatments are separately performed, and low invasive system can be provided with an endoscope.

2) A system can be constituted by additionally attaching the present system to an existing endoscope system.

While the luminescence source is provided on the control box 33 in this embodiment, the present invention is not limited thereto and, for example, an LED can be provided on the tip portion of the endoscope. In this case, a special effect that the system is more miniaturized can be obtained.

As the fourth embodiment of the system of the present invention, an embodiment in which a control box that is electrically connected to a temperature measuring apparatus and a light source apparatus is provided in any one of the first to third embodiments can be exemplified. Using such control box, the temperature is automatically controlled.

Hereinafter the present invention will be further explained in detail by the following Examples, but the present invention is not limited thereto.

### EXAMPLE 1

For two cases of malignant tumors formed in the oral cavity (sarcoma and malignant melanoma whose origin was unknown), photodynamic hyperthermic chemotherapy using ICG was performed. The two cases made good progress and no recurrence and metastasis were confirmed even after 1 year had passed.

Generally, a malignant tumor formed in the oral cavity easily causes topical recurrence and distant metastasis, and the prognosis thereof is not good. However, a good progress could be obtained where photodynamic hyperthermic chemotherapy (PHCT) using ICG (Diagnogreen, manufactured by Daiichi Pharmaceutical Co., Ltd.) was performed in a malignant tumor formed in the oral cavity in which manifestation of distant metastasis was not observed.

Case 1

A mixed-breed cat, castrated male, 8 years old, body weight 7.8 kg. The patient was brought to the hospital since a tumor was found on the mandibular incisor. Observation by the naked eyes at the first consultation: the tumor was projected in the form of a wart having a diameter of 5 mm on the front surface of the gingiva on the mandibular incisor.

Observation on the blood examination at the first consultation: AST was slightly increased.
X-ray observation during biopsy: invasion to the mandible was not observed by intraoral photographing. No metastatic focus was observed by simple chest radiography.
Histopathological diagnosis: sarcoma, the origin of which was unknown (resection biopsy: on the disease day 1). Resection state: unknown.
Treatment and course: since the recurrence of the tumor was observed up to the disease day 17, PHCT was carried out as follows on the disease day 46.

Cisplatin (Randa injection, 1 ml) was added to physiological saline (9 ml) whose pH had been adjusted to 5. ICG was dissolved in this solution, and 2 ml of which was topically administrated into the tumor tissue. A temperature sensor was set into the tissue, and a light was irradiated. As a light source apparatus, the apparatus shown in FIGS. 1 to 3 having a halogen lamp as the light source was used to irradiate light having a wavelength of from 600 to 1600 nm at an output of 5000 mW. The treatment period was 20 minutes per once. At the same time, the recurrent tumor was resected and asked to a reexamination. However, the diagnostic name remained unchanged, and the resection was diagnosed to be insufficient. Thereafter PHCT was performed three times at intervals of from 10 to 14 days. After 480 disease days had passed, no symptoms of recurrence and metastasis were observed.

Case 2

A golden retriever, male, 10 years old, body weight 31.0 kg. The patient was brought to a hospital since a black tumor was found on the oral surface of the mandible (under the tongue).

Observation on the blood examination at the first consultation: no abnormality was found.
Observation by the naked eyes during resection biopsy: the tumor existed on the center site being a little to the left canine tooth on the oral surface of the mandible. The diameter was about 20 mm and the color was black. The tumor was pedunculated and adhered to the mandibular bone. Since the tumor was very hard (as a cartilage), it was difficult to completely resect the tumor.
Diagnostic name of the pathologic tissue: malignant melanoma. Resection state was unknown.
Treatment and course: PHCT was performed as in Case 1 on the disease days 15, 23 and 30. On the disease day 87, slight protrusion of the site was reported.
On the disease day 94, resection biopsy was performed again and the fourth PHCT was performed. The diagnostic name of the pathologic tissue was malignant melanoma as well.
On the disease days 118 and 175, PHCT was performed.
On the disease day 217, the seventh PHCT was performed. At the same time, the treated tissue was collected and subjected to a pathological examination again. Since the disease was diagnosed as “fibrillization accompanied with deposition of melanin pigments”, the treatment was interrupted.

After 360 disease days had passed, no symptom of recurrence or metastasis was observed.

Since these two cases are of oral malignant tumors that are considered to be relatively liable to metastasis or recurrence, mandibular resection would have been applied in conventional cases. However, extended operation of the facial surface imposes great psychological resistance and burden of postoperative management on the guardians. Therefore, it is of great significance to be capable of controlling tumors or cancers by a low invasive method such as PHCT.

As described above, according to the present invention, tumors can be controlled by a low invasive method using a more simple apparatus and a more easy operation than those for hyperthermia, by performing topical warming by utilizing generation of heat by the exothermic reaction of the photosensitive dye agent to light.

What is claimed is:

1. A photodynamic hyperthermic chemotherapy of cancer, which comprises topically injecting a photosensitive dye agent in the form of an acidic liquid formulation into a tumor or cancer tissue, or a surgically removed site thereof of a patient, and irradiating the tissue or site with light having such an output wavelength that the photosensitive dye agent absorbs the light and generates heat.

2. The photodynamic hyperthermic chemotherapy according to claim 1, wherein the acidic liquid formulation is a liquid formulation of pH 4.0 to 6.8.

3. The photodynamic hyperthermic chemotherapy according to claim 1, wherein the acidic liquid formulation is an aqueous physiological saline solution.

4. The photodynamic hyperthermic chemotherapy according to claim 1, wherein the acidic liquid formulation is topically injected while keeping at 43 to 44° C.

5. The photodynamic hyperthermic chemotherapy according to claim 1, wherein the output wavelength is 600 to 1600 nm.

6. The photodynamic hyperthermic chemotherapy according to claim 1, wherein the output wavelength is 600 to 1300 nm.

7. The photodynamic hyperthermic chemotherapy according to claim 1, wherein the photosensitive dye agent is indocyanine green.

8. The photodynamic hyperthermic chemotherapy according to claim 1, wherein an anticancer agent is topically injected together with the photosensitive dye agent.

9. The photodynamic hyperthermic chemotherapy according to claim 1, wherein ethanol is topically injected together with the photosensitive dye agent.

10. The photodynamic hyperthermic chemotherapy according to claim 8, wherein the anticancer agent is cisplatin.

11. The photodynamic hyperthermic chemotherapy according to claim 8, wherein the anticancer agent is bleomycin.

12. The photodynamic hyperthermic chemotherapy according to claim 8, wherein the anticancer agent is paclitaxel.

13. The photodynamic hyperthermic chemotherapy according to claim 8, wherein the anticancer agent is carboplatin.

14. The photodynamic hyperthermic chemotherapy according to claim 1, wherein the tissue or site is irradiated plural times with light at an output of 5000 mW or more.

15. The photodynamic hyperthermic chemotherapy according to claim 1, wherein the patient is a human being.

16. The photodynamic hyperthermic chemotherapy according to claim 1, wherein the patient is an animal other than a human being.

17. An apparatus for photodynamic hyperthermic chemotherapy of cancer, which comprises a light source, and a probe having a light introducing portion capable of topically irradiating a tumor or cancer tissue, or a surgically removed site thereof of a patient with light derived from the light source by a continuous wave or a pulse wave at an output wavelength of 600 to 1600 nm and an output of 5000 mW or more.

18. The apparatus for photodynamic hyperthermic chemotherapy according to claim 17, wherein the light source is a halogen lamp.

19. A system for photodynamic hyperthermic chemotherapy of cancer by topically injecting an acidic liquid formulation comprising indocyanine green and an anticancer agent in the vicinity of a tumor or cancer tissue, or a surgically removed site thereof of a patient, and irradiating the tissue or site with light having such an output wavelength that indocyanine green is excited with causing an exothermic reaction, which comprises:
at least one light source of the light,
an irradiation means for irradiating the tissue or site with the light for exciting indocyanine green,
a warming means for warming the tissue or site by the exothermic reaction, and
a temperature measuring means for measuring the temperature of the tissue or site.

20. The system for photodynamic hyperthermic chemotherapy according to claim 19, wherein the light source is common to the irradiation means and the warming means.

21. The system for photodynamic hyperthermic chemotherapy according to claim 19, wherein the irradiation means and the warming means are combined.

22. The system for photodynamic hyperthermic chemotherapy according to claim 21, wherein the irradiation means comprises at least one irradiation port of light having a wavelength component for exciting indocyanine green, and a transmitting means for transmitting the light from the light source to the irradiation port; and the warming means shares the irradiation port and the transmitting means of the irradiation means, but irradiates a wavelength component that differs from the wavelength component for exciting indocyanine green to cause the exothermic reaction.

23. The system for photodynamic hyperthermic chemotherapy according to claim 22, wherein the irradiation port is a hard optical elongated element having a spherical tip and a rear end to which a portion connecting to the transmitting means which is an optical fiber is provided.

24. The system for photodynamic hyperthermic chemotherapy according to claim 23, wherein the irradiation port has an inner surface in a hemispherical shape and an irradiation aperture for the light introduced by the transmitting means at the bottom portion of the hemisphere.

25. The system for photodynamic hyperthermic chemotherapy according to claim 22, wherein the system includes an endoscope having a rigid or flexible tube through which the transmitting means of an optical fiber optically connected to the light source, at least one channel to allow entry of instruments or manipulators and a fiberscope for transmitting images are running; the tip portion of the transmitting means is optically connected to a light guide to constitute the irradiation port and the rear end of the transmitting means is connected to the light source; the tip portion of the endoscope has a cylindrical element, which is constituted by a transparent element having a longitudinal length corresponding to the focus distance of the objective lens of the fiberscope; apertures of the channel and the light guide are provided on the inner portion of the cylindrical element; and at least one thermocouples is provided on the tip portion of the cylindrical element.

26. The system for photodynamic hyperthermic chemotherapy according to claim 25, wherein the endoscope further has at least one channel for reducing air pressure in the channel and the cylindrical element.

27. The system for photodynamic hyperthermic chemotherapy according to claim 19, which has a circuit for controlling the output of the warming means based on a signal from the temperature measuring means.

28. The system for photodynamic hyperthermic chemotherapy according to claim 19, which further comprises a means for topically injecting the liquid formulation.
